SEQUENCE LISTING (7 sequences)

SEQ ID NO: 1 - protein, 9 aa, synthetic construct
  MOD_RES 8: note = X is azido lysine
CYIQNCPXG

SEQ ID NO: 2 - protein, 9 aa, synthetic construct
CYFQNCPRG

SEQ ID NO: 3 - protein, 14 aa, synthetic construct
AGCKNFFWKTFTSC

SEQ ID NO: 4 - protein, 21 aa, synthetic construct
CSCSSLMDKECVYFCHLDIIW

SEQ ID NO: 5 - protein, 19 aa, synthetic construct
IPRCDVGERCALKHGPRIG

SEQ ID NO: 6 - protein, 25 aa, synthetic construct
IPRCDVGERCALKHGPRIGRLCDCL

SEQ ID NO: 7 - protein, 38 aa, synthetic construct
IPRCDVGERCALKHGPRIGRLCDCLRGAACNTFFLRCL

(19) United States
(12) Patent Application Publication
(10) Pub. No.: US 2025/0042937 A1
(43) Pub. Date: Feb. 6, 2025

(54) RAPID, GREEN, DISULFIDE BOND FORMATION IN WATER USING DICYANOCOBINAMIDE

(71) Applicant: Syracuse University, Syracuse, NY (US)

(72) Inventors: Robert Doyle, Manlius, NY (US); Alyssa Spear, Cazenovia, NY (US)

(73) Assignee: Syracuse University, Syracuse, NY (US)

(21) Appl. No.: 18/792,216

(22) Filed: Aug. 1, 2024

Related U.S. Application Data
(60) Provisional application No. 63/530,156, filed on Aug. 1, 2023.

(51) Int. Cl. C07K 1/113; C07K 1/36; C07K 14/00

(52) U.S. Cl. CPC C07K 1/1133; C07K 1/36; C07K 14/001

(57) ABSTRACT

Methods and compositions are provided for oxidizing peptides and forming disulfide bonds. In some aspects, a rapid, green, and facile oxidation of peptides (e.g., cysteine containing peptides) is performed (e.g., to promote the formation of 1-3 or more disulfide bonds). In some aspects, methods are conducted in aqueous solutions, in air, utilizing a biocompatible corrin ring-containing compound (e.g., dicyanocobinamide). In some aspects, reaction times are under 1 hour and a simple one step removal of the catalyst can be performed.

### CROSS-REFERENCE TO RELATED APPLICATIONS

The present application claims priority under 35 U.S.C. § 119(e) to U.S. Provisional App. No. 63/530,156 filed on Aug. 1, 2023, the entire contents of which are incorporated herein by reference.

### STATEMENT REGARDING FEDERALLY SPONSORED RESEARCH AND DEVELOPMENT

This invention was made with government support under Grant No. DK128443 awarded by the National Institutes of Health (NIH). The government has certain rights in the invention.

### REFERENCE TO AN ELECTRONIC SEQUENCE LISTING

The contents of the electronic sequence listing (5234170001US00-SEQ-KUW.xml; Size: 7,346 bytes; and Date of Creation: Jul. 31, 2024) is herein incorporated by reference in its entirety.

### BACKGROUND OF THE INVENTION

### Field of the Invention

The present disclosure relates to methods for forming disulfide bonds in proteins, for example in proteins made using solid phase synthesis or other protein production techniques.

### Description of the Related Art

Solid-phase peptide synthesis (SPPS) is an indispensable tool for chemists to produce milligram to gram scale amounts of peptides and study their structure-activity relationships (SARs) to generate derivatives or analogues for new targeted therapeutics. Select multi-cysteine containing peptides require an extra synthetic step to achieve final three-dimensional structure, which influences their biological function and/or stability. The oxidation of a pair of cysteine residues to form a cystine produces a covalent bond and decreases the configurational entropy of the folded peptide. This process can be achieved via air oxidation, use of dimethyl sulfoxide (DMSO), hydrogen peroxide, iodine, or ferricyanide, to name a few. Many of the methods, however, require long reaction times, produce dimers, involve environmentally harmful organic solvents (which also limit hydrophilic peptide solubility), or result in modification of sensitive amino acid side chains (Tyr, Met, or Trp). Shi et al., demonstrated selective and rapid (30 min) formation of disulfide bonds using trans-[Pt(en)₂Cl₂]²⁺, however only dithiol peptides were studied and the catalyst is neither green nor commercially available. Laps et al., reported the ultrafast (˜3 min) formation of multiple disulfide bonds in peptides, however this methodology required multiple steps, multiple reagents, and multiple conditions (pH tuning) to facilitate this ‘step-wise’ folding strategy.

One approach for a rapid, aqueous based method to produce correct disulfide bonds post-SPPS that also allows facile purification involves the use of 10% aqueous DMSO. There is a need to move away from the polar aprotic solvent, which has slow reaction times (typically 2 days) and is not desirable for translation from in vitro to in vivo work in many pharmaceutical environments. Though DMSO has been used ubiquitously as a solvent for small hydrophobic drugs and as a control vehicle for a variety of biomedical studies, questions have been called on the suitability of the solvent in these contexts. DMSO has been reported to be cytotoxic at low doses (2-5%) in rat retinas, alter behaviour in aquatic species, and inactivate platinum-based drugs. It may also mask solubility issues that manifest when moving into saline or buffered aqueous solutions when moving to in vivo studies.

### BRIEF SUMMARY

Provided are methods for forming disulfide bonds in a peptide using a corrin complex, e.g., dicyanocobinamide (Cbi). In some embodiments, provided are methods for forming disulfide bonds in solid phase protein synthesis that use dicyanocobinamide (Cbi), which is a cheap, biocompatible, green and commercially available vitamin B₁₂ (B₁₂) precursor. In some embodiments, provided are methods for forming disulfide bonds in recombinantly synthesized peptides. In some aspects Cbi is readily prepared from B₁₂ using a simple 10-minute one-step method. In some aspects, the methods for forming disulfide bonds use a cobalt corrin complex. In some aspects, the methods for forming disulfide bonds use a cobalamin. In some aspects, the methods for forming disulfide bonds use a cobinamide. In one aspect, the present invention is a method of forming disulfide bonds in solid state peptide synthesis that involves the steps of providing a peptide having a cysteine residue, reacting the peptide with an amount of a corrin complex, e.g., dicyanocobinamide for a period of time until a disulfide bond is formed in the peptide. The amount of corrin complex, e.g., dicyanocobinamide may be between about 0.01 and about 2.0 molar equivalent relative to the peptide. In some embodiments, the amount of corrin complex, e.g., dicyanocobinamide is between about 0.06 and about 1.5 molar equivalent relative to the peptide. In some embodiments, the amount of corrin complex, e.g., dicyanocobinamide is about 1.0 molar equivalent relative to the peptide. In some embodiments, the peptide comprises about 7 amino acid residues to about 100 amino acids residues. In some embodiments, the peptide comprises about 9 amino acid residues to about 40 amino acids. In some embodiments, the method comprises the step of reacting the peptide with the amount of dicyanocobinamide which step comprises combining the peptide with the amount of dicyanocobinamide in a solution. In some embodiments, the solution is open to atmosphere. In some embodiments, the period of time is between about 20 minutes to about 2 hours. In some embodiments, the period of time is between about 20 minutes to about 1 hour. In some embodiments, the period of time is about 30 minutes. In some embodiments, the solution contains 4-(2-hydroxyethyl)-1-piperazineethanesulfonic acid. In some embodiments, the method further comprises purifying the peptide having the disulfide bond from the amount of corrin complex, e.g., dicyanocobinamide. In some embodiments, the step of purifying the peptide having the disulfide bond from the amount of corrin complex, e.g., dicyanocobinamide comprises chromatography, e.g., high-performance liquid chromatography. In some embodiments, the step of purifying the peptide having the disulfide bond from the amount of corrin complex, e.g., dicyanocobinamide comprises filtration, e.g., spin filtration. In another aspect, provided is a peptide having a disulfide bond formed by reacting the peptide with an amount of a corrin complex, e.g., dicyanocobinamide for a period of time until a disulfide bond is formed.

### BRIEF DESCRIPTION OF THE DRAWINGS

The present invention will be more fully understood and appreciated by reading the following Detailed Description in conjunction with the accompanying drawings, in which:

FIG. 1 is a schematic of (A) The structure of dicyanocobinamide (Cbi) and, (B) the sequence and disulfide bond connections of peptides screened (K [symbol] =azido lysine).

FIG. 2 is a graph of Cyclic Voltammogram of dicyanocobinamide (Cbi). Measurements were carried out in DMF at a scan rate of 100 mV s⁻¹ with Fc/Fc⁺ employed as an internal standard and are reported vs. Fc/Fc⁺.

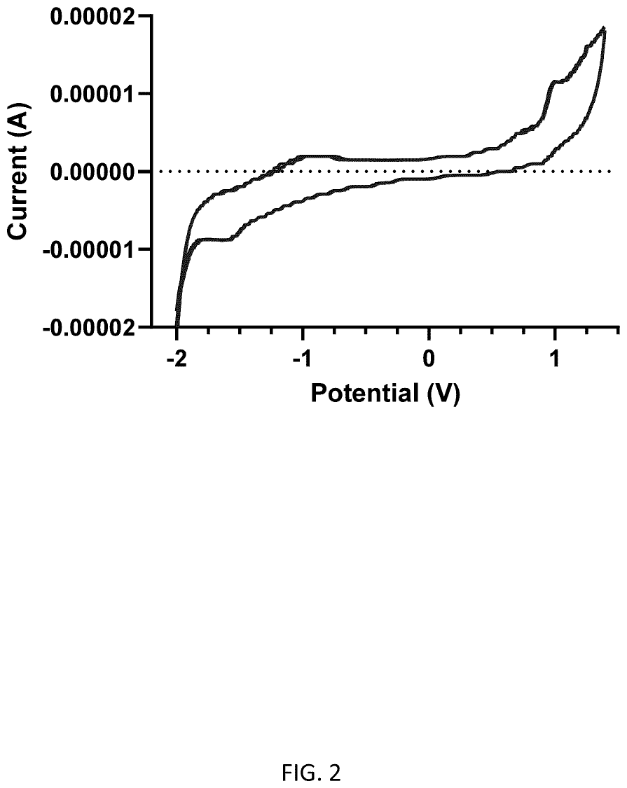

FIG. 3 are HPLC traces (280 nm) of OT reacted with 10% DMSO showing a retention time shift at 2880 min. HPLC method B was used with an Agilent ZORBAX 300SB-C8 (5 μm, 9.4×250 mm) column at 2 mL/min flow rate.

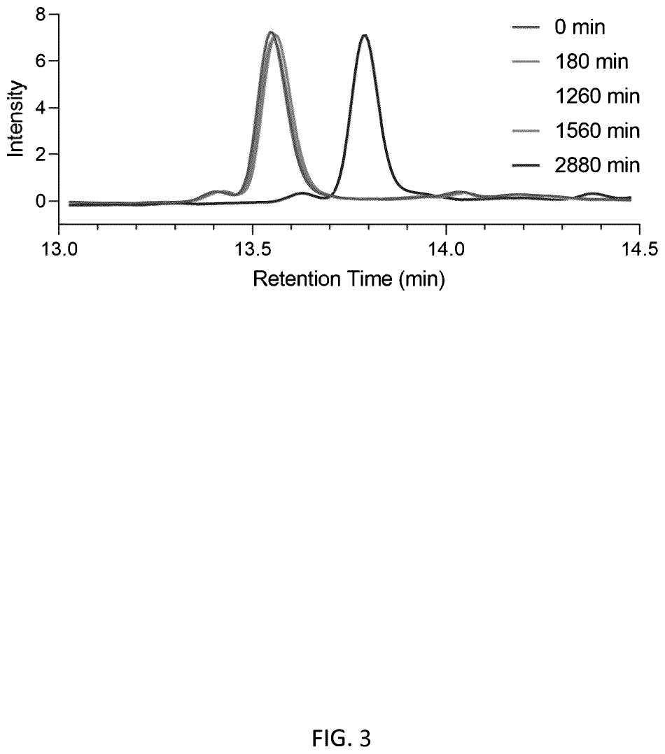

FIG. 4 are HPLC traces (280 nm) of OT reacted with 0.1 molar equivalences of Cbi showing a retention time shift at 1560 min. HPLC method B was used with an Agilent ZORBAX 300SB-C8 (5 μm, 9.4×250 mm) column at 2 mL/min flow rate.

FIG. 5 is an HRMS of (A) reduced OT with the expected mass of 1050 m/z, (B) oxidized OT obtained from reaction with Cbi for 1 hour with the expected mass of 1048 m/z, and (C) oxidized OT reacted with DTT with the expected mass of 1050 m/z.

FIG. 6 are HPLC traces (280 nm) of AVP upon reaction with Cbi showing an elution time shift from 6.494 min to 6.527 min in 90 min on method B.

FIG. 7 are HPLC traces (280 nm) of AVP upon reaction with Cbi in degassed H₂O on method B. These experiments were performed in a 2 mL auto sampling vial with a septum cap at room temperature. The reaction progressed to completion after 4 hours as opposed to 90 minutes when the reaction is open to air.

FIG. 8 is an HRMS of (A) reduced AVP with the expected mass of 543 m/z (z=2, 1086 parent mass), (B) oxidized AVP obtained from reaction with Cbi for 90 min with the expected mass of 542 m/z (z=2, 1084 parent mass), and (C) oxidized AVP reacted with DTT with the expected mass of 543 m/z (z=2, 1086 parent mass).

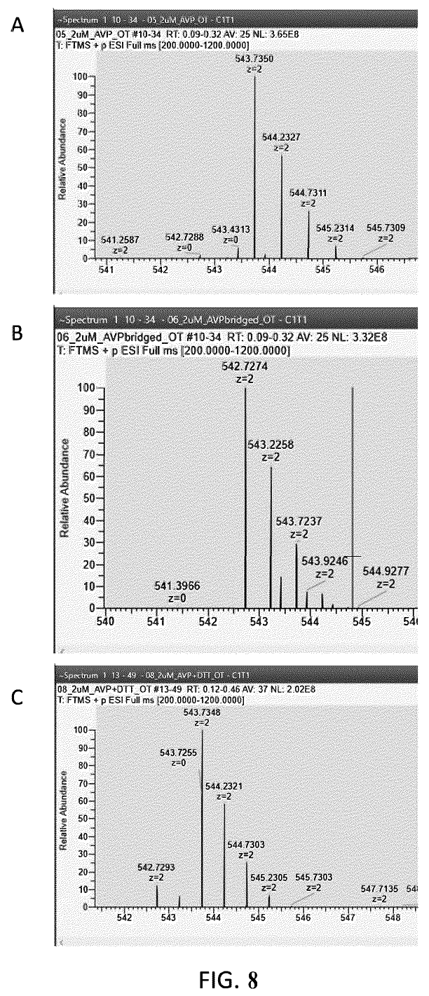

FIG. 9 is an HRMS of (A) SST reduced with the expected mass of 819.8825 m/z (z=2, 1637.764 monoisotopic mass), (B) oxidized SST obtained from reaction with Cbi with 1 hour with the expected mass of 818.844 m/z (z=2, 1635.749 monoisotopic mass), and (C) SST oxidized with DMSO for 48 hours with the expected mass of 818.875 m/z (z=2, 1635.749 monoisotopic mass)

FIG. 10 is an HRMS of reduced TrCART-1 full scan (left) and zoomed (right) with the appropriate mass of 2075.1505 (m/z 416.0301, z=5).

FIG. 11 is an HRMS of oxidized TrCART-1 obtained from reaction with Cbi for 1 hour full scan (left) and zoomed (right) with the appropriate mass of 2073.1416 (m/z 519.2854, z=4).

FIG. 12 is an HRMS of reduced TrCART-2 full scan (left) and zoomed (right) with the appropriate mass of 2077.6455 (m/z 556.2910, z=5).

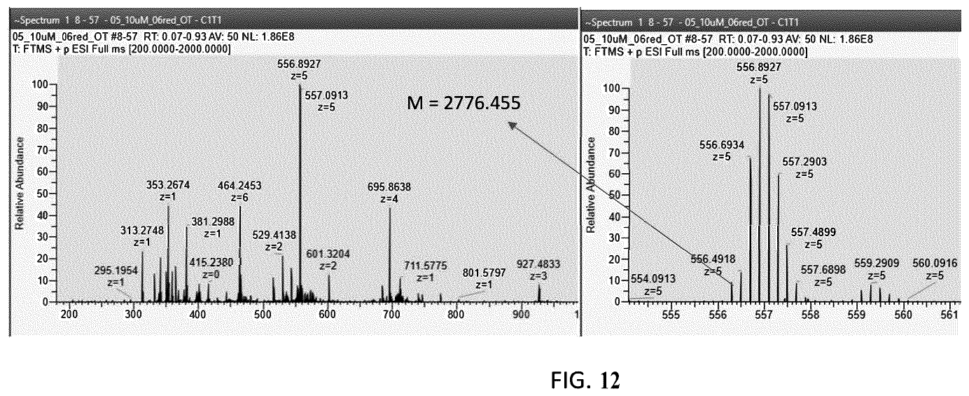

FIG. 13 is a schematic of predicted disulfide linkages and trypsin fragments of TrCART-2 (SEQ ID NO: 6). The sequence “LCDCL” corresponds to positions 21-25 of SEQ ID NO: 6. The sequence “CDVGER” corresponds to positions 4-9 of SEQ ID NO: 6. The sequence “CALK” corresponds to positions 10-13 of SEQ ID NO: 6. The sequence “IPR” corresponds to positions 1-3 of SEQ ID NO: 6. The sequence “HGPR” corresponds to positions 14-17 of SEQ ID NO: 6. The sequence “IGR” corresponds to positions 18-20 of SEQ ID NO: 6.

FIG. 14 is an HRMS of oxidized TrCART-2 obtained from reaction with Cbi for 1 hour and digested showing the detection of expected ionized trypsin fragments. The smaller fragments were not observed.

FIG. 15 is an HRMS of reduced TrCART-3 full scan (top) and zoomed (bottom) with the appropriate mass of 4227.1542 (m/z 705.5257, z=6).

FIG. 16 is a schematic of predicted disulfide linkages and trypsin fragments of TrCART-3 (SEQ ID NO: 7). The sequence “IPR” corresponds to positions 1-3 of SEQ ID NO: 7. The sequence “HGPR” corresponds to positions 14-17 of SEQ ID NO: 7. The sequence “IGR” corresponds to positions 18-20 of SEQ ID NO: 7. The sequence “CALK” corresponds to positions 10-13 of SEQ ID NO: 7. The sequence “GAACNTFFLR” corresponds to positions 27-36 of SEQ ID NO: 7. The sequence “CDVGER” corresponds to positions 4-9 of SEQ ID NO: 7. The sequence “LCDCLR” corresponds to positions 21-26 of SEQ ID NO: 7. The sequence “CL” corresponds to positions 24-25 of SEQ ID NO: 7.

FIG. 17 is a graph of electronic absorbance spectral changes recorded during Cbi (0.038 mM) reaction with OT (0.038 mM) in 2 mL of 20 mM HEPES (pH=7) at room temperature in a quartz cuvette equipped with a stir bar. Spectra were recorded at a cycle time of 20 min for 3 hours. Absorbance at 367 and 580 decreased (ΔAbs=0.053 and 0.023, respectively).

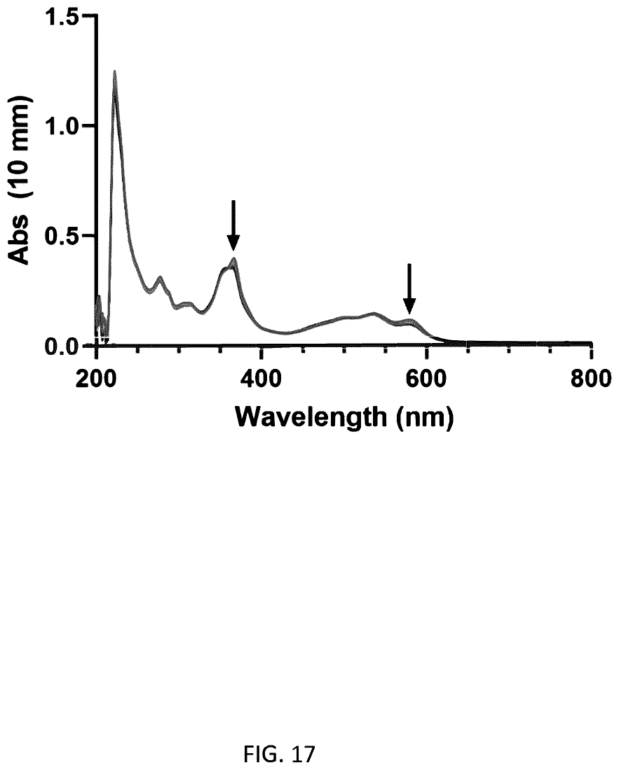

FIG. 18 is a schematic of a proposed mechanism of air-oxidation of cysteine residues to a cystine bond facilitated by Cbi.

The drawings are non-limiting disclosures related to embodiments described herein.

### DETAILED DESCRIPTION

Provided herein are methods using corrin complexes, e.g., Cbi, for forming one or more disulfide bonds in a peptide. In some aspects, provided is a methods of forming one or more disulfide bonds in a peptide. In some aspects, provided is a method of forming one or more disulfide bonds in solid state peptide synthesis. In some aspects, provided are methods of forming disulfide bonds in recombinantly synthesized peptides.

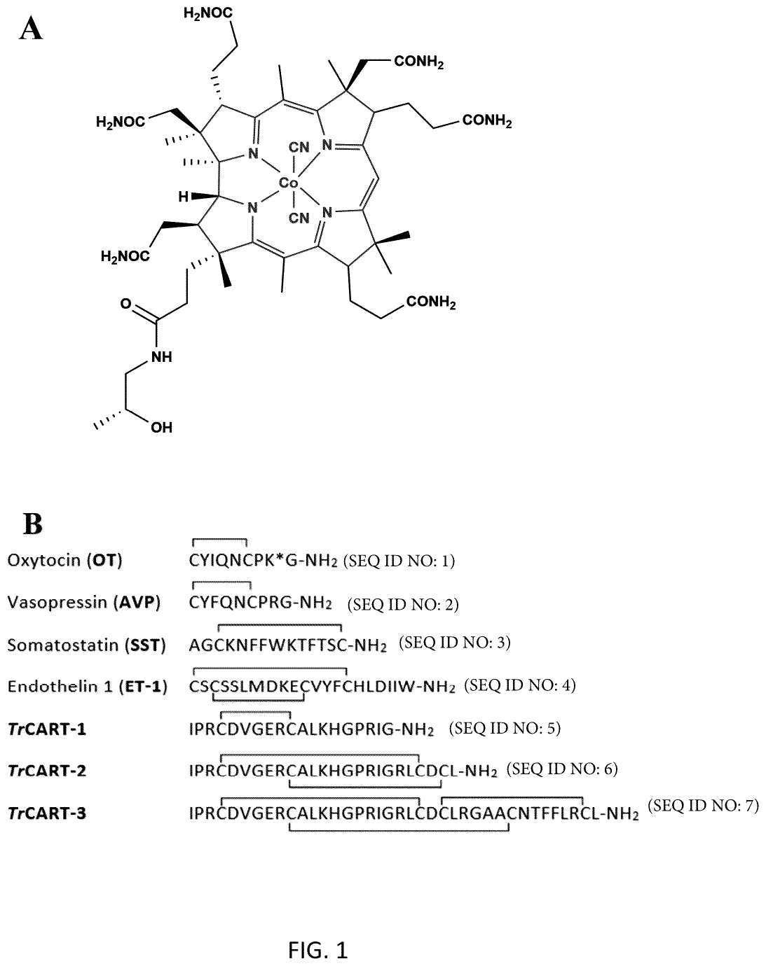

Referring to the figures, wherein like numerals refer to like parts throughout, there is seen in FIGS. 1(A and B), an approach for forming disulfide bonds in solid phase protein synthesis that uses dicyanocobinamide (Cbi), which is a cheap, biocompatible, green and commercially available vitamin B₁₂ (B₁₂) precursor. The cobalt center of B₁₂ can access three oxidation states giving rise to electrophilic, radical, and nucleophilic behavior each with different ligand-accepting capabilities and reduction potentials with influence from its 2,3-dimethylbenzimidazole base-on/-off forms and modification to the corrin macrocycle. A number of excellent reviews and perspectives are available on B₁₂ chemistry, with recent interest in applications ranging from dicarbofunctionalization of bromoalkenes to transition metal analogues for the study of B₁₂-dependant processes to regioselective ring opening of epoxides. In contrast, there is a limited understanding of Cbi, which has been primarily studied as an antidote for cyanide poisoning or as a scavenger for other poisons like methyl mercaptan or hydrogen sulphide. Over the course of the 1960s to 1980s, cobalamins and cobinamides were shown to oxidize simple thiols such as 2-mercaptoethanol and dithioerythritol. Of note was the observation that Cbi catalysed the aerobic oxidation orders of magnitude faster than B₁₂ (191 and 0.003 s⁻¹, respectively). Despite these long-known reports, no one has developed a method for disulfide bond oxidation via Cbi to prepare functional peptides.

In some aspects, a method comprises providing a peptide comprising a cysteine residue (e.g., two or more cysteine residues). In some aspects, a peptide comprises at least about seven amino acid residues. In some aspects, a peptide comprises about seven amino acid residues to about 100 amino acids residues. The term “about,” as used herein, refers to a value that is similar to a stated value and within a range of values that fall within 20% in either direction (greater than or less than). In some aspects, a peptide comprises about 8 amino acid residues to about 98 amino acids residues or about 10 to about 96 amino acids, about 12 to about 90 amino acids, about 14 to about 85 amino acids, about 16 to about 80 amino acids, about 18 to about 75 amino acids, about 20 to about 70 amino acids, about 22 to about 65 amino acids, about 24 to about 60 amino acids, about 26 to about 55 amino acids, about 28 to about 50 amino acids, or about 30 to about 45 amino acids. In some aspects, a peptide comprises about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, about 25 amino acids, about 26 amino acids, about 27 amino acids, about 28 amino acids, about 29 amino acids, about 30 amino acids, or more amino acids.

In some aspects, a peptide comprises two thiol-containing residues (e.g., two or more thiol-containing residues). In some aspects, a peptide comprises four thiol-containing residues (e.g., four or more thiol-containing residues). In some aspects, a peptide comprises six thiol-containing residues (e.g., six or more thiol-containing residues). In some aspects, a peptide comprises 8, 10, 12, 14, 16, 18, 20, 22, 24, 26, 28, 30 or more thiol-containing residues.

In some aspects, a peptide comprises two cysteine residues (e.g., two or more cysteine residues). In some aspects, a peptide comprises four cysteine residues (e.g., four or more cysteine residues). In some aspects, a peptide comprises six cysteine residues (e.g., six or more cysteine residues). In some aspects, a peptide comprises 8, 10, 12, 14, 16, 18, 20, 22, 24, 26, 28, 30 or more cysteine residues.

For example, the peptides used in the present study have a mix of lengths and numbers of cysteine residues to test the versatility of Cbi catalysis of disulfide oxidation, as seen in FIG. 1B. Regioselectivity of disulfide bonding in cysteine-rich peptides presents a challenge for oxidation methods due to the possibility of scrambling and subsequent misfolding. The present disclosure describes the utilization of Cbi to catalyse the air-oxidation of the peptides with fast reaction times, in water, with facile purification, and with discussion of regioselectivity.

In some aspects, provided are methods comprising the use of a corrin complex to form one or more disulfide bonds in a peptide. “Corrin complex” refers to a metal coordination complex of a compound comprising a corrin ring:

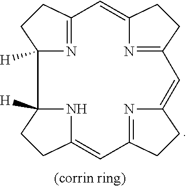 [SMILES: C1=C2/CCC(=N2)/C=C2/CC[C@@H](N2)[C@H]2CCC(=N2)/C=C2/CCC/1=N2]

In some aspects, the corrin complex is a cobalt corrin complex. In certain aspects, the corrin complex is a cobalamin. In certain aspects, the corrin complex is a cobinamide.

In some aspects, provided are methods comprising the use of a corrin complex, e.g., dicyanocobinamide (Cbi) to form one or more disulfide bonds in a peptide. In some aspects, a method comprises the use of a vitamin B 12 corrin complex to form one or more disulfide bonds in a peptide. In some aspects, a method provided comprises the use of a corrin complex, e.g., Cbi in ambient air. In some aspects, a method provided comprises the use of a corrin complex, e.g., Cbi under air restricted conditions. In some aspects, a method provided comprises the use of a corrin complex, e.g., Cbi in degassed water.

In some aspects, provided is a method of forming one or more disulfide bonds in a peptide, the method comprising reacting a peptide with an amount of a corrin complex, e.g., Cbi for about 1 min to about 5 hours, or about 2 minutes to about 4.5 hours, about 5 minutes to about 4 hours, about 10 minutes to about 3.5 hours, about 15 minutes to about 3 hours, about 20 minutes to about 2.8 hours, about 25 minutes to about 2.6 hours, about 30 minutes to about 2.4 hours, about 35 minutes to about 2.2 hours, about 40 minutes to about 2 hours, about 45 minutes to about 1.8 hours, about 47 minutes to about 1.6 hours, about 50 minutes to about 1.4 hours, about 55 minutes to about 1.2 hours, or about 1 hour. In some aspects, the method comprises reacting a peptide with an amount of a corrin complex, e.g., Cbi for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, about 75 minutes, about 76 minutes, about 77 minutes, about 78 minutes, about 79 minutes, about 80 minutes, about 81 minutes, about 82 minutes, about 83 minutes, about 84 minutes, about 85 minutes, about 86 minutes, about 87 minutes, about 88 minutes, about 89 minutes, or about 90 minutes.

In some aspects, provided is a method of forming one or more disulfide bonds in a peptide, the method comprising reacting a peptide with a corrin complex, e.g., Cbi, which method can be performed at room temperature or any other suitable temperature, e.g., at about 15° C. to about 35° C. In some aspects, provided is a method of forming one or more disulfide bonds in a peptide, the method comprising reacting a peptide with a corrin complex, e.g., Cbi, wherein the amount of corrin complex, e.g., Cbi is between about 0.01 molar and about 10 molar equivalent relative to the peptide, or about 0.02 to about 9, about 0.03 to about 8, about 0.04 to about 7, about 0.05 to about 6, about 0.06 to about 5, about 0.07 to about 4, about 0.08 to about 3, about 0.09 to about 2, or about 0.1 to about 1 molar equivalent relative to the peptide. In some aspects, provided is a method of forming one or more disulfide bonds in a peptide, the method comprising reacting a peptide with a corrin complex, e.g., Cbi, wherein the amount of corrin complex, e.g., Cbi is about 0.01 to about 2 molar, 0.06 to about 1.5 molar, or about 1 molar equivalent relative to the peptide.

In some aspects, provided is a method of forming one or more disulfide bonds in a peptide, the method comprising reacting a peptide with a corrin complex, e.g., Cbi, wherein the amount of corrin complex, e.g., Cbi is about 0.01 molar, about 0.02 molar, about 0.04 molar, about 0.06 molar, about 0.08 molar, about 0.1 molar, about 0.2 molar, about 0.3 molar, about 0.4 molar, about 0.5 molar, about 0.6 molar, about 0.7 molar, about 0.8 molar, about 0.9 molar, or about 1 molar equivalent relative to the peptide.

As a model substrate, a modified oxytocin (OT) was used for detailed screening of Cbi. OT incorporates an azido-modified lysine at position 8 and is frequently synthesized. An equimolar amount of peptide was combined with 2 mg Cbi in 2 mL of 20 mM HEPES solution (pH=7). The reaction was done in a 20 mL clear glass vial open to air with stirring. After 30 minutes of reaction, HPLC was performed to detect a retention time shift of 7.629 to 7.506 min. Oxidized peptide was collected in stoichiometric yield as determined by HPLC peak area. To validate the HPLC results, the eluted peptide was collected, lyophilized, and redissolved in an aqueous solution of dithiothreitol (DTT, 10 mM), a strong reducing agent. The retention time was observed to revert to that of the reduced peptide (7.629 min).

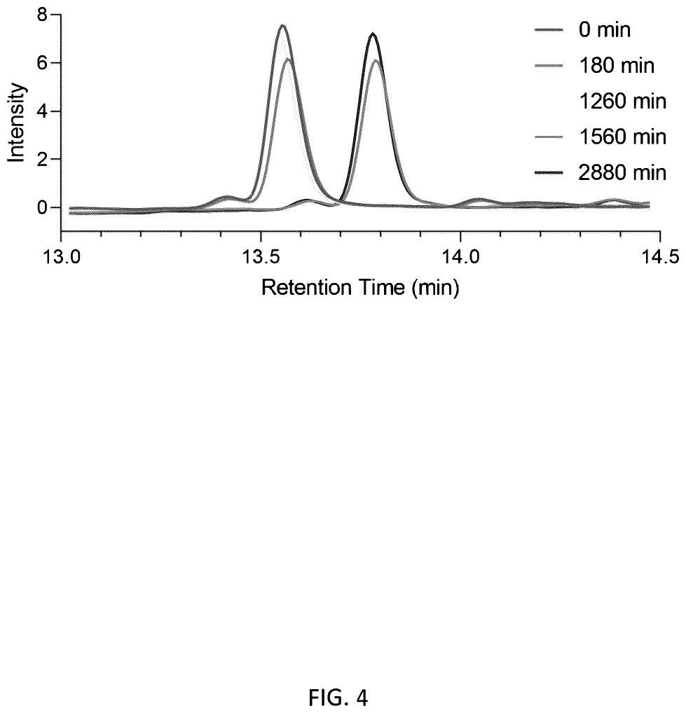

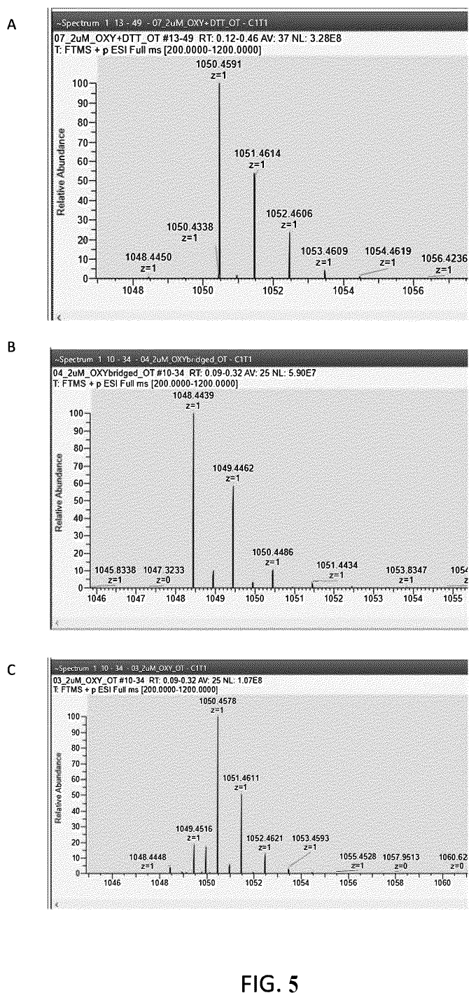

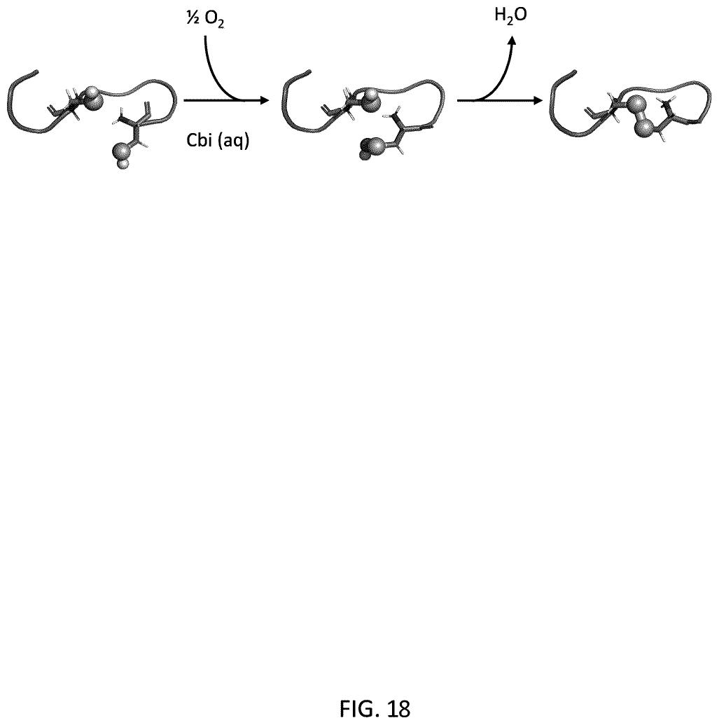

Purification was achieved by simple HPLC methods as described and shown herein for all peptides screened (FIG. 4). Separation based on molecular weight using commercially available spin filters was also shown, thus allowing for purification of Cbi from peptide by simple centrifugation only (see below and FIG. 18). Lyophilized powders of all purified reduced and oxidized peptides, as well as DTT-treated peptides, were subject to HRMS for confirmation (FIG. 5). The appropriate masses were observed for OT (reduced=1050.4578 m/z; oxidized=1048.4439 m/z; z=1) and the loss of two hydrogens indicative of oxidation. The DTT treated peptide also displayed the expected mass of the reduced peptide (1050.4591 m/z).

To determine the necessity of Cbi as metal catalyst, Co(II)C12 was employed as an analogue of the reduced metal centre. Co(II)C12 and peptide were combined at equimolar concentrations in 1 mL H₂O and the retention time of the peptide was tracked as described previously. Co(II)C12 did not oxidize the peptides under the conditions used. Furthermore, when B₁₂ was introduced as a reagent under the same conditions, oxidation was not observed over the course of 48 hours, suggesting a strong influence from the axial ligands. Additionally, side products were detected especially at extended reaction times of 24-48 hours.

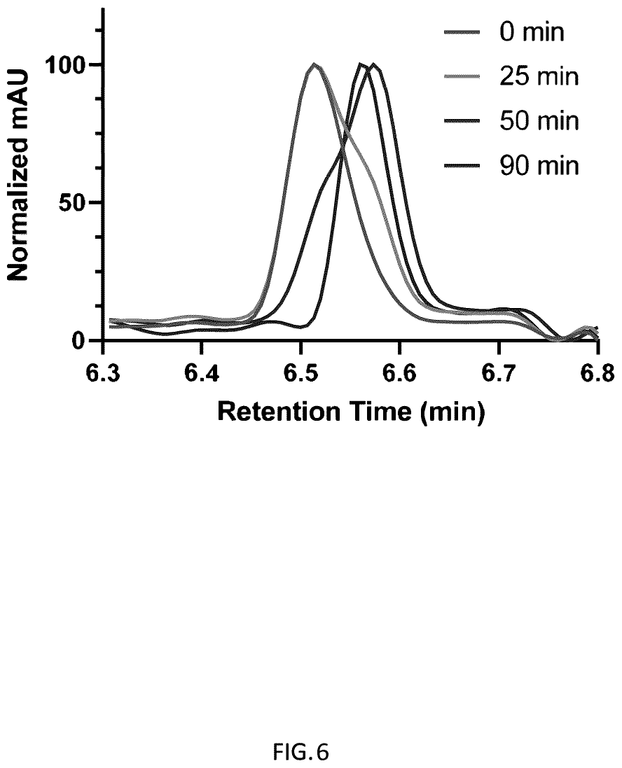

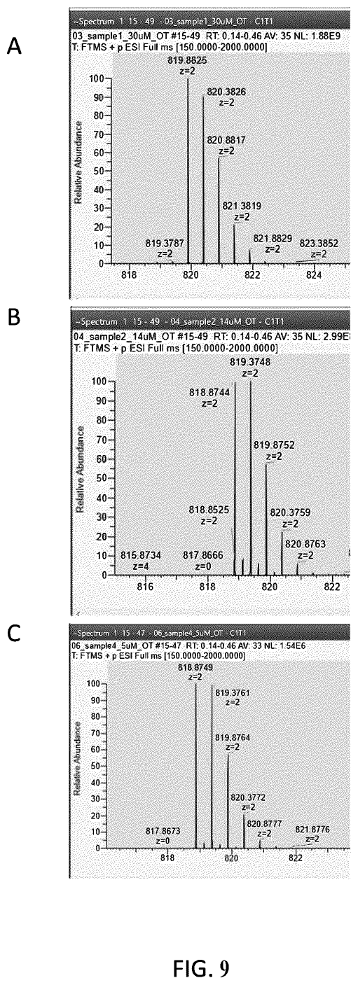

Cbi was then tested as a catalyst for oxidative disulfide formation in a library of peptides with varying lengths and number of disulfides (Table 1 below). Full peptide HPLC chromatograms and mass spectra can be found in the supplemental information. The single disulfide containing peptides consisted of OT, vasopressin (AVP), and somatostatin (SST). OT produced the sharpest line peaks, distinct shift in elution times (7.629 to 7.506 min), and shortest conversion time of 30 min. AVP, while similar in sequence to OT, had broader line peaks and took 90 min to convert, exemplified by a slight elution time shift of 6.494 to 6.527 (FIG. 6). SST also produced sharp line peaks but did not show any shift in elution time over the course of oxidation with either DMSO or Cbi. Rather than determining time of oxidation via HPLC, reactions were allowed to run over 48 hours, purified, lyophilized and subject to HRMS. SST displayed the appropriate mass for its oxidized form in 1 hour upon reaction with Cbi and 48 hours with DMSO (FIG. 9). The double disulfide containing peptide endothelin-1 (ET-1) was also studied and found to oxidize after 1 hour of reaction with Cbi. Commercial controls of AVP, SST, and ET-1 were bought in to further validate the HPLC retention times.

TABLE 1
Disulfide bond formation of peptides.

| | | Mol Equiv. of | Conversion |
|---|---|---|---|
| Reagent | Peptide | Reagent | Time (min) |
| DMSO | OT | — | 2880 |
| | AVP | — | 2880 |
| | SST | — | 2880 |
| | ET-1 | — | 2880 |
| | TrCART-1 | — | 1440 |
| | TrCART-2 | — | 2880 |
| | TrCART-3 | — | 2880 |
| Cbi | OT | 1 | 30 |
| | OT | 0.5 | 50 |
| | OT | 0.1 | 1560 |
| | AVP | 1 | 90 |
| | AVP | 1 | 243^{a} |
| | SST | 1 | 60 |
| | ET-1 | 1 | 60 |
| | TrCART-1 | 1 | 60 |
| | TrCART-2 | 1 | 60 |
| | TrCART-3 | 1 | 60 |
| Co(II)Cl | OT | 1 | 2880 |
| cyano-B₁₂ | OT | 1 | npo |

All reactions were performed open to air at a 2 mg scale. DMSO was used at 10% in aqueous solution. ^{a}Reaction performed in air free conditions with degassed water under argon. npo=no product observed at 2880 min.

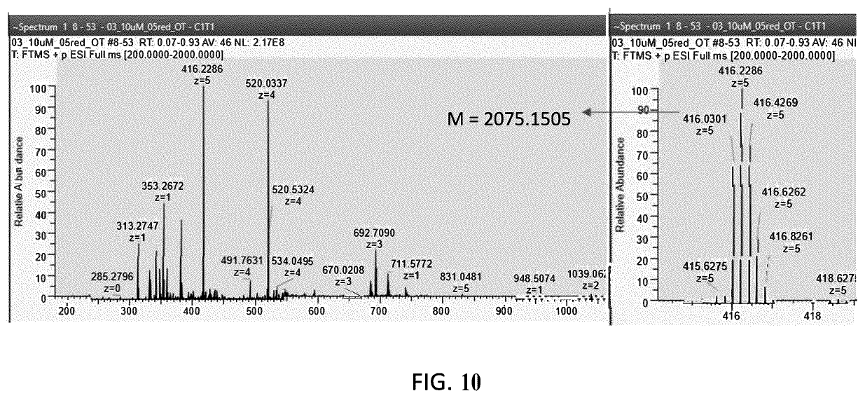

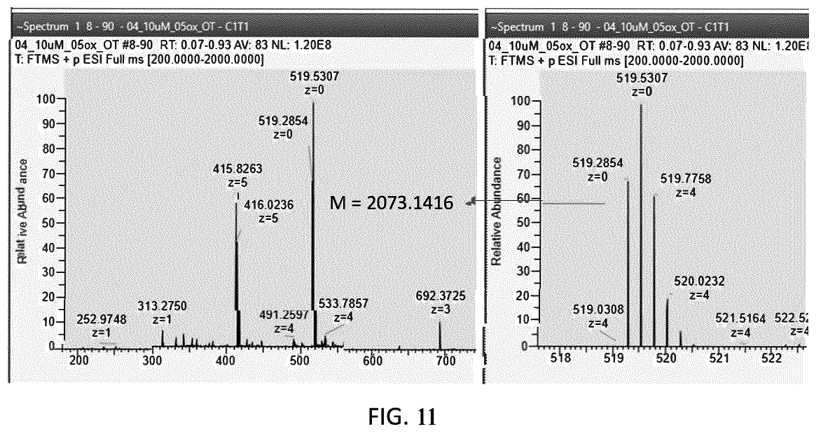

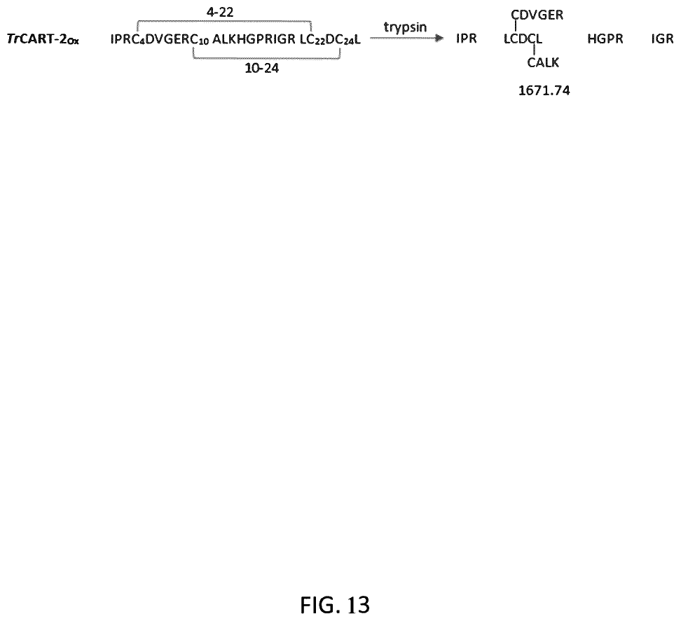

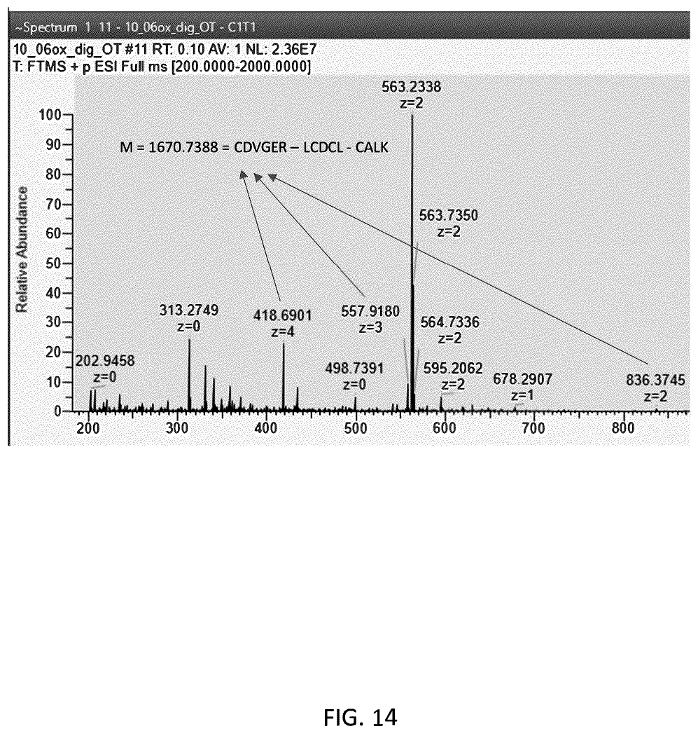

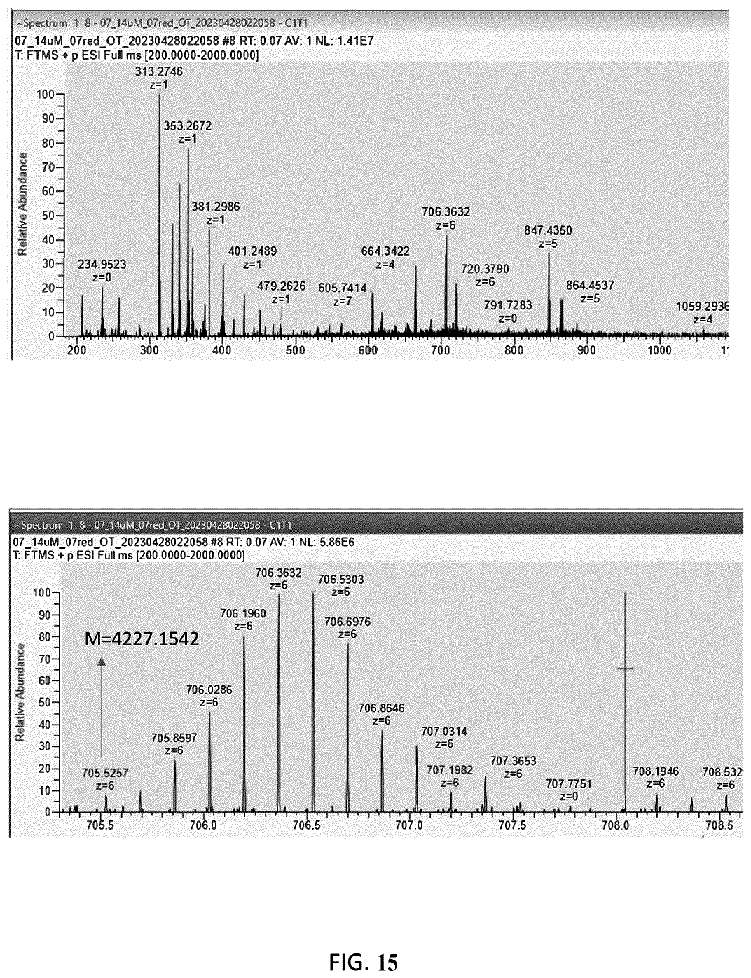

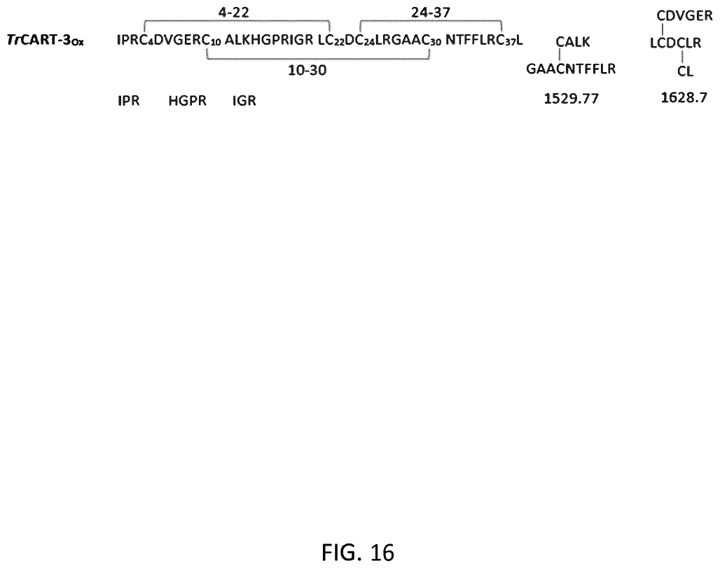

TrCART-(1-3) are a series of cocaine and amphetamine related transcript (CART) analogues derived from the Takifugu rubripes sequence. Each iteration of the analogue peptides incorporates a larger portion of the sequence and an increasing amount of disulfide bonds. TrCART-(1-3) were oxidized by Cbi within an hour while DMSO required 24-48 hours (FIGS. 10, 11, 13-16). TrCART-2 and 3 were subject to tryptic digest to determine the bound cysteines. TrCART-2 was predicted to contain disulfides at C4-22 and C10-24, the latter being the appropriate linkage for the native human CART (hCART). After digestion, the ionized species of the major fragment was detected (M=1670.7388; m/z 418, z=4; 557, z=3; 836, z=2) (FIGS. 13-14). TrCART-3 was predicted to have disulfide linkages at C4-22, C10-30, and C24-37, all corresponding to the folding pattern of hCART and supported by HRMS. The digested fragments of 1628.7 (543.9301, z=3) and 1529.77 (765.8832, z=2) were observed (FIG. 16). In both cases, the hydrophobicity of the peptide led to precipitation and faint signals during HRMS and trypsin digestion, especially for the fully oxidized peptides lacking free thiols.

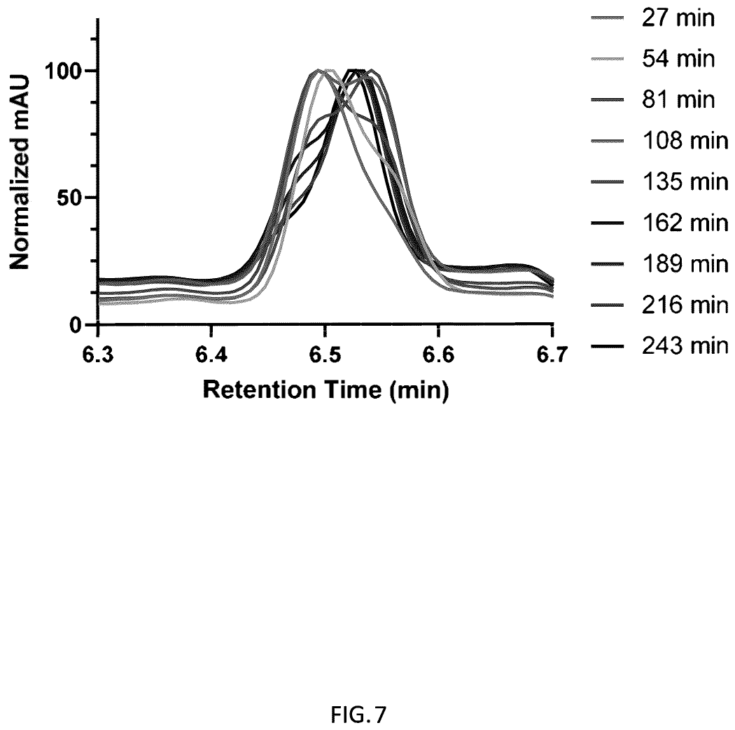

Two putative mechanisms offered up by literature are (1) direct coordination of a thiol to the Co(III) center and (2) air-oxidation catalyzed by Cbi as consistent with literature detailing nonenzymatic protein folding. Spectroscopic tracking of the reaction to obtain evidence Co—S bind formation revealed only minor spectral changes over the course of 3 hours and was therefore not consistent with direct binding of the peptide with Cbi. As a result, the role of oxygen with Cbi was explored. Initially, the catalysis in water was repeated and degassed by three rounds of freeze-pump-thaw to remove oxygen. AVP was then reacted with Cbi, resulting in a conversion time of 4 hours as opposed to 90 minutes in air, introduced when the auto sampling needle punctured the septum over recurring assay time-points (FIG. 7). Oxidation via atmospheric 02, as opposed to Cbi itself, is further supported when considering reduction potentials. Cbi (FIG. 18) was measured as having a reduction potential of −1.5 V vs Fc^{+/0} in DMF, while O₂ has a reported potential of +0.6 V under the same reference and solvent. It is hypothesized then that Cbi is playing a role in activating molecular oxygen to form a sulfenic acid moiety on the peptide followed by a condensation reaction between the —SH and —SOH groups resulting in the bridged peptide and H₂O (FIG. 18), though more experiments are needed to support this mechanism of action.

Finally, the functionality post Cbi oxidation was confirmed by screening one of the peptides, OT, both free and after conjugation to B₁₂ at the oxytocin receptor with equipotent agonism recorded (K_{D}=4.39 and 4.63 nM, respectively).

In conclusion, Cbi is a powerful catalyst for the oxidation of cysteine residues in peptides synthesized via SPPS. Cbi can be synthesized from vitamin B₁₂ or purchased commercially. When synthesized from B₁₂, assuming 92% literature yield, and running at a 2 mg scale, these reactions currently cost $0.27 per reaction with respect to Cbi. The system is one step, one pot, one hour, requiring water and air and offers facile routes to purification. Catalysis with Cbi was shown to impart regioselectivity in TrCART-2 and 3, implying that the cobalt-bound construct was able to stabilize or direct folding towards the thermodynamic product within the short time span of 1 hour. The method described herein provides chemists with a rapid, one-step, aqueous route to achieve the oxidized forms of peptides with a commercially available, green oxidant.

### Examples

### Materials and Instrumentation

Dimethylformamide (DMF), dichloromethane (DCM), diethyl ether, trifluoroacetic acid (TFA), triisopropyl silane (TIPS), N,N′-diisopropylcarbodiimide (DIC), piperidine, NaCl, a-cyano-4-hydroxy-cinnamic acid (CHCA), and HPLC grade acetonitrile (MeCN) were purchased from Sigma Aldrich (Milwaukee, WI, USA). Fmoc-protected amino acids, Oxyma Pure, and ProTide rink-amide resin were purchased from CEM (Matthews, NC, USA). Dithiothreitol (DTT) was purchased from MP Biomedicals, LLC (Solon, OH, USA).

Deionized water was prepared in-house and filtered through a Nalgene Rapid-Flow 0.2 μm PES vacuum filter prior to use. 50 mM HEPES stock solution was prepared by dissolving 11.915 HEPES free acid and 6.83 g NaCl in a total volume of 1 L of deionized water. The solution was diluted to 20 mM and the pH was adjusted to 7 with KOH.

Peptides were synthesized in-house on a microwave assisted CEM Liberty Blue peptide synthesizer and cleaved from the support resin using a CEM Razor. Reverse-phase high performance liquid chromatography (RP-HPLC) was performed on an Agilent 1100 Series instrument with an Agilent ZORBAX 300SB-C8 (5 μm, 9.4×250 mm), Agilent Eclipse XDB-C18 column (3.5 μm, 4.6×100 mm), or Agilent Eclipse XDB-C18 column (5 μm, 4.6×150 mm). MALDI-TOF MS was conducted on a Bruker microflex with an MSP 96 polished steel BC target and CHCA matrix. High resolution mass spectrometry (HRMS) was performed on a Thermo Orbitrap Fusion Lumos at the SUNY Upstate Proteomics & Mass Spectrometry Core Facility.

For spin filtration studies, Amicon Ultra—15 centrifugal filters (Ultracel—3K) were used (Merck Millipore Ltd., Cork, Ireland).

### HPLC Methods

All HPLC analyses were done in reverse phase with a gradient system of 0.1% TFA in water (solvent A) and 0.1% TFA in MeCN (solvent B). The diode array detector was set to 280 nm and 360 nm to detect the peptides and Cbi, respectively. In the case a peptide did not have an aromatic residue to detect at 280 nm, 220 nm was used to detect peptide bonds.

Method A was used for peptide purification and developed on an Agilent ZORBAX 300SB-C8 column (5 μm, 9.4×250 mm) using the following gradient: 10%-75% ACN over 15 min, 95% ACN for 5 min, 10% ACN for 5 min. Methods B-D were used for analytical studies. Method B was developed on an Eclipse Plus C18 column (3.5 μm, 4.6×100 mm) using the following gradient: 1-70% ACN for 15 min, 95% ACN for 5 min, 1% ACN for 5 min. Method C was developed on an Agilent Eclipse XDB-C18 column (5 μm, 4.6×150 mm) using the following gradient: 18% ACN for 5 min, 18-60% ACN for 5 min, 95% ACN for 2.5 min, 18% ACN for 2.5 min. Method D was developed on an Agilent Eclipse XDB-C18 column (5 μm, 4.6×150 mm) using the following gradient: 20% ACN for 5 min, 20-75% ACN for 5 min, 95% ACN for 2.5 min, 20% ACN for 2.5 min.

### Peptide Synthesis

Peptides were synthesized following solid-phase chemistry at a 0.025 mmol scale on rink amide resin on an automatic peptide synthesizer. Fmoc-protected amino acids were prepared at 0.2 M in DMF. The activator and activator base used for coupling were Oxyma Pure (0.25 M) and DIC (0.125 M), respectively. The deprotection reagent used between couplings was 20% piperidine in DMF.

After synthesis, the resin-bound peptides were transferred to the cleavage instrument with DCM, the DCM was removed under vacuum filtration, and the deprotection/cleavage solution was added (95% TFA, 2.5% TIPS, and 2.5% H₂O) and left to incubate for 40-45 min at 40° C. The peptides were precipitated with cold (−20° C.) diethyl ether and centrifuged for 10 min at 4,000 rpm to obtain a crude pellet.

Purification was achieved by RP-HPLC using method A. The peptide was then flash frozen with liquid nitrogen and lyophilized to a dry powder. Purity traces were obtained using methods B-D and >90% purity was ensured before further experimentation.

### Cbi Synthesis

Cbi was prepared in-house as described by Gryko et al.,¹ however, it is also commercially available from Sigma-Aldrich. Cyanocobalamin (100 mg, 0.074 mmol), NaCN (13 mg, 0.25 mmol), and EtOH (6.6 mL) were added to a microwave reaction vessel with a magnetic stir bar and sealed with a cap. The vessel was heated to 120° C. for 10 min using 300 W. The mixture was transferred to a flask using EtOH and reduced to minimal volume by rotary evaporation. The concentrated mixture was then loaded onto a normal phase flash chromatography cartridge and eluted using an isocratic method of MeOH in EtOAc (2:1 v:v) at a flow rate of 5 mL/min. The major violet fraction was isolated, dried by rotary evaporation, dissolved in iPrOH, and vacuum filtered through a celite plug to remove any excess cyanide and silica. The filtrate was then dried, redissolved in water, and lyophilized. ¹H NMR (400 MHz, D₂O) δ 5.891 (s, 1H), 3.907 (m, J=5.95 Hz, 1H), 3.839 (d, J=8.23, 1H), 3.748 (d, J=10.44, 1H), 3.654 (q, J=7.11, 2H), 3.572 (q, J=7.08 Hz, 1H), 3.404 (dd, J=7.00, 4.78, 1H), 3.312-3.151 (3H, overlapped), 2.914-2.857 (m, 1H), 2.748-2.712 (m, 4H), 2.600-2.380 (6H, overlapped), 2.316-2.231 (14H, overlapped), 2.148-2.069 (m, 4H), 2.030-1.943 (m, 1H), 1.915 (s, 3H), 1.891-1.744 (m, 3H), 1.683 (s, 3H), 1.532 (s, 3H), 1.489 (s, 3H), 1.430 (s, 3H), 1.312 (s, 3H), 1.184 (t, J=7.12 Hz, 7H), 1.151 (d, J=6.36 Hz, 3H); UV-vis (H₂O) λ 276, 312, 367, 503, 539, 580 nm; MS (MALDI-TOF) calculated for C₄₈H₇₃CoN₁₁O₈ ([M−2CN+H]⁺) 991.11, found 990.72; HPLC t_{R} values on method B: 1.839 and 2.105 min; method C: 1.989 and 2.837; and method D: 1.852 and 2.282.

### Peptide Oxidation Reactions

DMSO reactions. 10% DMSO was prepared in 20 mM HEPES solution. A peptide sample (0.0019 mmol) was dissolved in the 10% DMSO in a 20 mL clear glass scintillation vial with a stir bar. The solution was stirred at room temperature and aliquots were taken periodically for HPLC analysis to observe a retention time shift.

Cbi reactions. Cbi (2 mg, 0.0019 mmol) and peptide (0.0019 mmol) were combined in 2 mL of 20 mM HEPES solution in a 20 mL clear glass scintillation vial with a stir bar. The solution was allowed to stir at room temperature open to air and aliquots were taken periodically for HPLC analysis to observe a retention time shift. In the case no retention time shift was observed, the solution was allowed to stir for 1 hour after which the peptide was purified via HPLC and immediately lyophilized and subsequently submitted for HRMS.

Air restricted reaction. 20 mM HEPES was degassed through three rounds of freeze-pump-thaw. Cbi (1 mg, 0.95 μmol) and peptide (0.95 μmol) were combined in 1 mL of 20 mM HEPES solution in a 2 mL autosampler vial and the headspace was purged with argon. The sample was submitted for periodic HPLC analysis to observe a retention time shift.

TABLE 2
Peptide MW and amount (mg) used per
reaction (equivalent to 0.0019 mmol).

| | Reduced MW | Amt. used per |
|---|---|---|
| Peptide | (g/mol) | reaction (mg) |
| OT | 1050 | 2.0 |
| AVP | 1086 | 2.1 |
| SST | 1639 | 3.1 |
| ET-1 | 2495 | 4.8 |
| TrCART-1 | 2076 | 4.0 |
| TrCART-2 | 2780 | 5.3 |
| TrCART-3 | 4234 | 8.1 |

### TrCART-3 Purification Via Spin Column Filtration

Cbi (1042 g/mol, 2 mg, 0.0019 mmol) and crude TrCART-3 (4234 g/mol, 8.1 mg, 0.0019 mmol) were combined in 5 mL of 18% ACN in ultrapure water and pipetted into the spin column (3000 MWCO) taking care to not touch the filters. The column was centrifuged at 4000 rpm for 25 min. The filtrate was collected, and an additional 5 mL of solvent was added to the top chamber and gently pipetted up and down to clear the filters. Washing and filtration was repeated until the peptide solution ran clear (5 washes) and an HPLC trace was taken to verify complete filtration of Cbi.

An HPLC purity trace (280 nm) of reduced OT showed 90% purity at an elution time of 7.673 min on method B. HPLC traces (280 nm) of OT reacted with 1 molar equivalent of Cbi on method C showed a complete retention time shift at 30 min. HPLC traces (280 nm) of OT reacted with 0.5 molar equivalences of Cbi on method B showed a complete retention time shift at 50 min.

HPLC traces (280 nm) of OT on method B showed (A) a full range trace of oxidized OT with a retention time of 7.550 min, (B) a zoomed in trace of 1 mM OT treated with 10 mM of DTT upon initial mixing, and (C) a zoomed in trace of 1 mM OT treated with 10 mM of DTT after 25 min of mixing. The oxidized OT peak at 7.506 min shrinks and the reduced OT peak at 7.629 min grows in as the DTT reduces the OT.

HPLC traces (280 nm) of OT reacted with 1 molar equivalent of CoCl₂ on method C showed a retention time shift at 1500 min but incomplete conversion out to 2880 min. A side product was observed at a retention time of ˜4.6 min. HPLC traces (280 nm) of OT reacted with B₁₂ via method B over 2880 min showed a large band at 6 min that is B₁₂ while OT was observed at ˜7.63 min consistent with its reduced state. Side-products were detected at ˜5.2 and ˜8.0 min but their identities could not be confirmed.

An HPLC purity trace (280 nm) of reduced AVP showed 99% purity at an elution time of 6.396 min on method B. An HPLC trace of AVP commercial standard showed the oxidized peptide retention time at 6.631 min on method B. HPLC traces (280 nm) of AVP in 10% DMSO over the course of 48 hours on method B showed little conversion of reduced AVP (6.41 min) to oxidized AVP (6.67 min).

An HPLC purity trace (280 nm) of SST showed an elution time of 9.557 min and purity of 98.9% on method C. HPLC traces (280 nm) of SST oxidation with Cbi for 1 hour showed no shift in elution time over the course of oxidation on method C. HPLC traces (280 nm) of SST oxidation with DMSO for 1 hour showed no shift in elution time over the course of oxidation on method C. An HPLC trace (280 nm) of SST reacted with Cbi or DMSO for 1 hour and a commercial SST standard all showed the same retention time shift of ˜9.5 min on method C.

An HPLC purity trace of oxidized ET-1 obtained from reaction with Cbi for 1 hour and commercial standard on method D showed elution times of 9.377 and 9.446 minutes, respectively. A TrCART-1 HPLC purity trace on method C showed an elution time of 4.927 min with 98% purity on method C. HPLC traces of TrCART-1 in 10% DMSO over 48 hours showed complete oxidation after 24 hours on method C. MALDI-ToF MS traces of TrCART-1 in 10% DMSO over 48 hours showed complete oxidation after 24 hours. An HPLC purity trace of reduced TrCART-2 showed an elution time of 8.543 min on method C using an Eclipse Plus C18 column (3.5 μm, 4.6×100 mm).

HPLC traces (top) of crude TrCART-2 in 10% DMSO over 48 hours method C with an Eclipse Plus C18 column (3.5 μm, 4.6×100 mm) showed that the peak at ˜7.6 was eluted and the mass, via MALDI-ToF MS (bottom), was found to be that of oxidized TrCART-2 (2776 m/z). An HPLC purity trace (220 nm) of reduced TrCART-3 showed 94% purity at an elution time of 9.946 min on method C. HPLC traces (220 nm) of TrCART-3 reacted with DMSO over the course of 48 hours showed that, from 1 to 18 hours, no change in retention time was observed. At 48 hours, the HPLC profile was observed to change, however mixed products are suggested by method C.

An HRMS of oxidized TrCART-3 obtained from reaction with Cbi for 1 hour and digested showed the detection of expected ionized trypsin fragments. HPLC traces (top, 220 nm; bottom, 360 nm) of TrCART-3 before mixing with Cbi on method C as compared to HPLC traces (top, 220 nm; bottom, 360 nm) of TrCART-3 after mixing with Cbi and 5 rounds of spin column filtration showing no Cbi at the detection wavelength of 360 nm.

What is claimed is:

1. A method of forming one or more disulfide bonds in a peptide, the method comprising the steps of:
providing a peptide having two or more cysteine residues;
reacting the peptide with an amount of a corrin complex for a period of time until a disulfide bond is formed in the peptide.

2. The method of claim 1, wherein the corrin complex is dicyanocobinamide.

3. The method of claim 1, wherein the amount of corrin complex is between about 0.01 and about 2.0 molar equivalent relative to the peptide.

4. The method of claim 1, wherein the amount of corrin complex is between about 0.06 and about 1.5 molar equivalent relative to the peptide.

5. The method of claim 1, wherein the amount of corrin complex is about 1.0 molar equivalent relative to the peptide.

6. The method of claim 1, wherein the peptide comprises about 7 amino acid residues to about 100 amino acids residues.

7. The method of claim 6, wherein the peptide comprises about 9 amino acid residues to about 40 amino acid residues.

8. The method of claim 1, wherein the step of reacting the peptide with the amount of the corrin complex comprises combining the peptide with the amount of corrin complex in a solution.

9. The method of claim 8, wherein the solution is open to atmosphere.

10. The method of claim 1, wherein the period of time is between about 20 minutes to about 2 hours.

11. The method of claim 10, wherein the period of time is about 20 minutes to about 1 hour.

12. The method of claim 11, wherein the period of time is about thirty minutes.

13. The method of claim 8, wherein the solution contains 4-(2-hydroxyethyl)-1-piperazineethanesulfonic acid.

14. The method of claim 1, further comprising purifying the peptide having the disulfide bond from the amount of corrin complex.

15. The method of claim 14, wherein purifying is a one-step removal of the corrin complex.

16. The method of claim 14, wherein purifying comprises high-performance liquid chromatography.

17. The method of claim 14, wherein purifying comprises spin filtration.

18. The method of claim 1, wherein the peptide is a solid-state synthesized peptide.

19. The method of claim 1, wherein the peptide is a recombinantly synthesized peptide.

20. A peptide having a disulfide bond formed by the method of claim 1.
